(19) United States
(12) Patent Application Publication
(10) Pub. No.: US 2020/0148633 A1
(43) Pub. Date: May 14, 2020

(54) METHOD FOR PREPARING HYDROGEN BIS(FLUOROSULFONYL)IMIDE AND METHOD FOR PREPARING LITHIUM BIS(FLUOROSULFONYL)IMIDE

(71) Applicant: Jiujiang Tinci Advanced Materials Co., Ltd., Jiujiang (CN)

(72) Inventors: Jingwei ZHAO, Jiujiang (CN); Yong XIN, Jiujiang (CN); Anle SUN, Jiujiang (CN); Chenglong ZHU, Jiujiang (CN); Lei XU, Jiujiang (CN)

(21) Appl. No.: 16/744,265

(22) Filed: Jan. 16, 2020

Related U.S. Application Data
(63) Continuation-in-part of application No. PCT/CN2018/110188, filed on Oct. 15, 2018.

(30) Foreign Application Priority Data
Jul. 31, 2018 (CN) 201810855656.X

(51) Int. Cl. C07C 303/34

(52) U.S. Cl. CPC C07C 303/34

(57) ABSTRACT

A method for preparing hydrogen bis(fluorosulfonyl)imide including contacting sulfonyl fluoride with hexamethyl disilazane in an organic solvent. The disclosure also provides a method for preparing lithium bis(fluorosulfonyl)imide (LiFSI). The method includes contacting sulfonyl fluoride with hexamethyl disilazane in an organic solvent and yielding hydrogen bis(fluorosulfonyl)imide; and contacting hydrogen bis(fluorosulfonyl)imide with a lithium compound and yielding lithium bis(fluorosulfonyl)imide.

### CROSS-REFERENCE TO RELATED APPLICATIONS

This application is a continuation-in-part of International Patent Application No. PCT/CN2018/110188 with an international filing date of Oct. 15, 2018, designating the United States, now pending, and further claims priority benefits to Chinese Patent Application No. 201810855656.X filed Jul. 31, 2018. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND

The disclosure relates to the field of chemical synthesis, and more particularly to a method for preparing hydrogen bis(fluorosulfonyl)imide (HFSI) and lithium bis(fluorosulfonyl)imide (LiFSI).

HFSI is a fluorine containing compound and in recent years, the interests in HFSI have been concentrated on the preparation of its metallic salts, ionic liquids, eutectic mixtures, which are used in electronics.

LiFSI has been studied as a conducting salt for nonaqueous liquid electrolytes for lithium-ion batteries.

Conventional preparation methods of HFSI and LiFSI include fluorinating hydrogen bis(chlorosulfonyl)imide using SbCl₅, TiCl₄, SnCl₄, MoCl₅, as a catalyst. The method produces HCl as gaseous byproduct.

### SUMMARY

The disclosure provides a method for preparing hydrogen bis(fluorosulfonyl)imide and method for preparing lithium bis(fluorosulfonyl)imide.

Specifically, the method for preparing hydrogen bis(fluorosulfonyl)imide comprises contacting sulfonyl fluoride with hexamethyl disilazane in an organic solvent, and the reaction process is as follows:

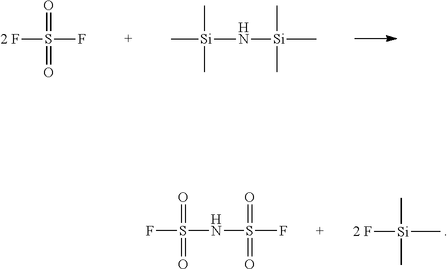

The organic solvent can be an ester, an amide, or a nitrile; the ester can comprise ethyl acetate and butyl acetate; the amide can comprise N, N-dimethylformamide, N, N-Dimethylacetamide, and N-methylpyrrolidone; and the nitrile can comprise acetonitrile and propiononitrile.

The method can comprise dissolving the sulfonyl fluoride in the organic solvent, and adding the hexamethyl disilazane to a mixture of the sulfonyl fluoride and the organic solvent; and the usage amount of the organic solvent can be at least 0.1 L per mole of hexamethyl disilazane.

Contacting the sulfonyl fluoride with the hexamethyl disilazane can be carried out at the temperature of between 30 and 110° C. for 2-10 hours.

The molar ratio of the sulfonyl fluoride to the hexamethyl disilazane can be between 2:1 and 5:1.

A byproduct of trimethylfluorosilane can be produced, and the method can further comprise contacting the trimethylfluorosilane with ammonia gas to yield the hexamethyl disilazane.

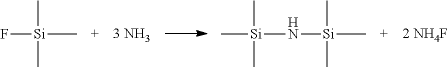

The disclosure also provides a method for preparing lithium bis(fluorosulfonyl)imide (LiFSI), the method comprising:

contacting sulfonyl fluoride with hexamethyl disilazane in an organic solvent, thereby yielding hydrogen bis(fluorosulfonyl)imide; and

contacting hydrogen bis(fluorosulfonyl)imide with a lithium compound, thereby yielding lithium bis(fluorosulfonyl)imide.

The lithium compound can be selected from the group consisting of Li, LiH, LiNH₂, LiF, LiOH, LiHCO₃, Li₂CO₃, or a mixture thereof.

The organic solvent can be a polar solvent selected from the group consisting of dimethyl carbonate, diethyl carbonate, methylethyl carbonate, propylene carbonate, vinyl carbonate, methyl acetate, propyl acetate, isopropyl acetate, ethyl acetate, butyl acetate, isobutyl acetate, ether, propyl ether, isopropyl ether, butyl ether, isobutyl ether, tetrahydrofuran, methyltetrahydrofuran, dioxane, ethylene glycol dimethyl ether, ethylene glycol diethyl ether, acetone, butanone Methyl isobutyl ketone, cyclopentanone, cyclobutanone, N, N-dimethylformamide, N, N-Dimethylacetamide, N-methylpyrrolidone, dimethyl sulfoxide, acetonitrile, propiononitrile, or a mixture thereof; and an addition amount of the organic solvent is at least 0.1 L per mole of hexamethyl disilazane.

The molar ratio of the sulfonyl fluoride to lithium of the lithium compound can be between 1:1 and 1:2.

Contacting hydrogen bis(fluorosulfonyl)imide with a lithium compound can be carried out at a temperature of between 0 and 20° C. for 1-10 hours, particularly, between 0 and 5° C.

The method can comprise mixing the lithium compound and the organic solvent, cooling a mixture of the lithium compound and the organic solvent to between 0 and 2° C., dropwise adding hydrogen bis(fluorosulfonyl)imide to the mixture, resting the mixture and the hydrogen bis(fluorosulfonyl)imide at a temperature of between 0 and 2° C. for 1-5 hours, filtering and collecting a supernatant, concentrating the supernatant, adding a polar or nonpolar solvent to a resulting concentrated supernatant thereby yielding a solid lithium bis(fluorosulfonyl)imide, filtering and drying the solid lithium bis(fluorosulfonyl)imide.

The supernatant can be concentrated to be 1.2-1.5 times of the hydrogen bis(fluorosulfonyl)imide by weight, and then the weak polar or nonpolar solvent is added, thereby precipitating the solid lithium bis(fluorosulfonyl)imide.

The polar or nonpolar solvent can be a halogenated hydrocarbon solvent, alkane solvent, halogenated aromatic hydrocarbon solvent; the halogenated hydrocarbon solvent comprises dichloromethane and dichloroethane; the alkane solvent comprises n-hexane, cyclohexane and n-heptane, and the halogenated aromatic hydrocarbon solvent comprises toluene, ethylbenzene and chlorobenzene; and an addition amount of the polar or nonpolar solvent is 1-5 times that of the solid lithium bis(fluorosulfonyl)imide by weight.

The hydrogen bis(fluorosulfonyl)imide and lithium bis(fluorosulfonyl)imide of the disclosure can be used for preparation of lithium-ion battery electrolyte and ultracapacitor.

The method employs existing industrial raw materials to synthesize hydrogen bis(fluorosulfonyl)imide in one step, no fluoridation involved, no corrosive gas produced, and no transition metal salts as a catalyst required. Thus, the method can reduce the difficulty of product separation and purification, and improve the reaction yield and product purity. In addition, the by-product of the method can be easily and quickly recycled; and the solvent used in the synthesis process of hydrogen bis(fluorosulfonyl)imide can be directly reused.

Likewise, hydrogen bis(chlorosulfonyl)imide (HClSI) can be synthesized by using hexamethyl disilazane and sulfonyl chloride according to the similar methods and conditions of the disclosure, and then the HClSI is fluorated to yield HFSI; or the HClSI as a raw material directly contacts LiF to yield LiFSI.

### DETAILED DESCRIPTION OF THE EMBODIMENTS

To further illustrate, embodiments detailing a method for preparing hydrogen bis(fluorosulfonyl)imide and method for preparing lithium bis(fluorosulfonyl)imide are described below. It should be noted that the following embodiments are intended to describe and not to limit the disclosure.

The disclosure provides a method for preparing hydrogen bis(fluorosulfonyl)imide, comprising contacting sulfonyl fluoride with hexamethyl disilazane in an organic solvent. The reaction process is as follows:

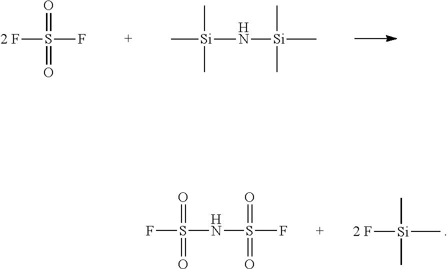

The method is easy to operate; the products are easy to separate and purify; the products have high purity and yield, no environmental pollution. The method for the disclosure overcome the disadvantages of conventional methods, such as complicated operation, low yield, environmental pollution caused by toxic reagents and fluorine-containing gas reagents, difficult purification of products, etc.

Specifically, the method comprises dissolving the sulfonyl fluoride in the organic solvent, and slowly adding the hexamethyl disilazane to a mixture of the sulfonyl fluoride and the organic solvent.

Specifically, the organic solvent is an ester, an amide, or a nitrile; the ester comprises ethyl acetate and butyl acetate; the amide comprises N, N-dimethylformamide, N, N-Dimethylacetamide, and N-methylpyrrolidone; and the nitrile comprises acetonitrile and propiononitrile. The usage amount of the organic solvent is at least 0.1 L per mole of hexamethyl disilazane, particularly 0.1-20 L, and more particularly 0.1-10 L.

Specifically, contacting the sulfonyl fluoride with the hexamethyl disilazane is carried out at a temperature of between 30 and 110° C., particularly between 70 and 100° C., for 2-10 hours.

Specifically, the molar ratio of the sulfonyl fluoride to the hexamethyl disilazane is between 2:1 and 5:1, particularly between 2.1:1 and 3:1.

The disclosure also provides a method for preparing lithium bis(fluorosulfonyl)imide, the method comprising:

contacting sulfonyl fluoride with hexamethyl disilazane in an organic solvent, thereby yielding hydrogen bis(fluorosulfonyl)imide (HFSI); and

contacting hydrogen bis(fluorosulfonyl)imide with a lithium compound, thereby yielding lithium bis(fluorosulfonyl)imide (LiFSI).

Specifically, the lithium compound is selected from the group consisting of Li, LiH, LiNH₂, LiF, LiOH, LiHCO₃, Li₂CO₃, or a mixture thereof.

When the lithium compound is lithium, the reaction is as follows:

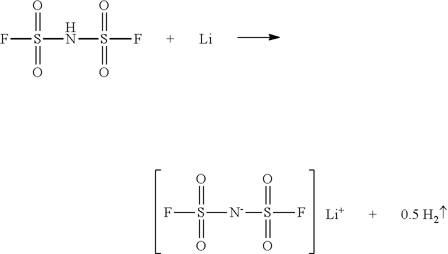

When the lithium compound is LiH, the reaction is as follows:

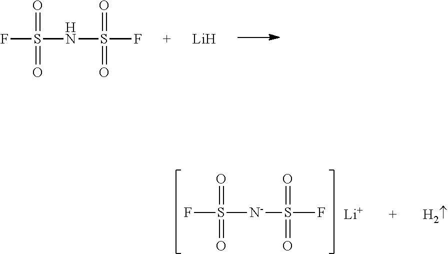

When the lithium compound is LiNH₂, the reaction is as follows:

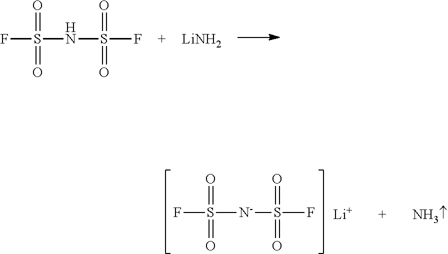

When the lithium-containing compound is a basic compound such as LiF, LiOH, LiHCO₃ or Li₂CO₃, HFSI reacts with the lithium-containing compound in acid-base neutralization to generate LiFSI.

The reaction is carried out in a polar solvent.

The polar solvent selected from the group consisting of dimethyl carbonate, diethyl carbonate, methylethyl carbonate, propylene carbonate, vinyl carbonate, methyl acetate, propyl acetate, isopropyl acetate, ethyl acetate, butyl acetate, isobutyl acetate, ether, propyl ether, isopropyl ether, butyl ether, isobutyl ether, tetrahydrofuran, methyltetrahydrofuran, dioxane, ethylene glycol dimethyl ether, ethylene glycol diethyl ether, acetone, butanone Methyl isobutyl ketone, cyclopentanone, cyclobutanone, N, N-dimethylformamide, N, N-Dimethylacetamide, N-methylpyrrolidone, dimethyl sulfoxide, acetonitrile, propiononitrile, or a mixture thereof; and an addition amount of the organic solvent is at least 0.1 L per mole of hexamethyl disilazane, particularly 0.1-20 L, and more particularly 0.1-10 L.

The molar ratio of the sulfonyl fluoride to lithium of the lithium compound is between 1:1 and 1:2, particularly between 1:1 and 1:1.2.

Specifically, contacting hydrogen bis(fluorosulfonyl)imide with a lithium compound is carried out at a temperature of between 0 and 20° C. for 1-10 hours, particularly between 0 and 5° C. for 1-3 hours. In the reaction process, the reaction system is cooled and the reactant is dropwise added.

The method comprises mixing the lithium compound and the organic solvent, cooling a mixture of the lithium compound and the organic solvent to between 0 and 2° C., dropwise adding hydrogen bis(fluorosulfonyl)imide to the mixture at the temperature of below 5° C., resting the mixture and the hydrogen bis(fluorosulfonyl)imide at a temperature of between 0 and 2° C. for 1-3 hours, filtering and collecting a supernatant, concentrating the supernatant, adding a weak polar or nonpolar solvent to a resulting concentrated supernatant thereby yielding a solid lithium bis(fluorosulfonyl)imide, filtering and drying the solid lithium bis(fluorosulfonyl)imide.

Specifically, the supernatant is concentrated to be 1.2-1.5 times of the hydrogen bis(fluorosulfonyl)imide by weight, and then the weak polar or nonpolar solvent is added, thereby precipitating the solid lithium bis(fluorosulfonyl)imide.

Specifically, the polar or nonpolar solvent is a halogenated hydrocarbon solvent, alkane solvent, halogenated aromatic hydrocarbon solvent; the halogenated hydrocarbon solvent comprises dichloromethane and dichloroethane; the alkane solvent comprises n-hexane, cyclohexane and n-heptane, and the halogenated aromatic hydrocarbon solvent comprises toluene, ethylbenzene and chlorobenzene; and an addition amount of the polar or nonpolar solvent is 1-5 times that of the solid lithium bis(fluorosulfonyl)imide by weight.

In the preparation process of hydrogen bis(fluorosulfonyl)imide, a byproduct of trimethylfluorosilane is produced. The method further comprises contacting the trimethylfluorosilane with ammonia gas to yield the hexamethyl disilazane, which is recycled and returns to the preparation process of HFSI. The reaction process is as follows:

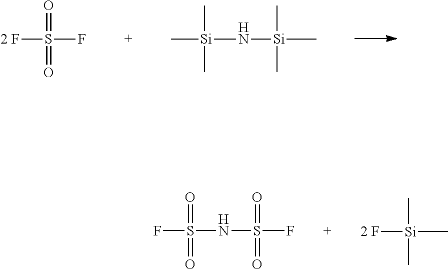

Specifically, the preparation of hexamethyl disilazane from trimethylfluorosilane is as follows: trimethylfluorosilane is added to a stainless-steel autoclave and stirred. NH₃ is added to the autoclave and the pressure of the autoclave is maintained at 0.1-0.2 megapascal, the temperature at 40-50° C. 0.5-2 hours later, the autoclave is cooled to below 10° C. Water below 10° C. is added to the autoclave to dissolve NH₄F. The supernatant is crude product of hexamethyl disilazane, which is dried and rectified to yield a final product comprising 99.0% of hexamethyl disilazane.

The disclosure is further described in combination with examples, where Examples 1-3 relate to preparation of hydrogen bis(fluorosulfonyl)imide, and Examples 4-6 relate to preparation of lithium bis(fluorosulfonyl)imide using the hydrogen bis(fluorosulfonyl)imide prepared in Examples 1-3.

### Example 1

150 mL of anhydrous acetonitrile and 76.5 g of sulfonyl fluoride were added to a 500-mL autoclave. At room temperature, 40.35 g of hexamethyl disilazane was slowly pumped into the autoclave. Thereafter, the autoclave was heated to 90° C. and maintained for 3 hours, and then unreacted sulfonyl fluoride and the byproduct trimethylfluorosilane were recycled through pressure distillation. The solvent (that is, anhydrous acetonitrile) was recycled through vacuum distillation and the final product of hydrogen bis(fluorosulfonyl)imide was obtained by distillation. The hydrogen bis(fluorosulfonyl)imide was 44.3 g, with a yield of 98%. The recycled sulfonyl fluoride and solvent directly returned to the reaction process, and the byproduct trimethylfluorosilane was used to prepare hexamethyl disilazane. Specifically, 43.8 g of produced trimethylfluorosilane was added to an autoclave and stirred. NH₃ was added to the autoclave and the pressure of the autoclave was maintained at 0.1-0.2 megapascal, the temperature at 40-50° C. 0.5-2 hours later, the autoclave was cooled to below 10° C. Water below 10° C. was added to the autoclave to dissolve NH₄F. The supernatant was crude product of hexamethyl disilazane, which was dried and rectified to yield a final product comprising 99.0% of hexamethyl disilazane. 36.4 g of hexamethyl disilazane was obtained, with a yield of 90%.

### Example 2

100 mL of N,N-dimethylformamide and 51.0 g of sulfonyl fluoride were added to a 500-mL autoclave. At room temperature, 40.35 g of hexamethyl disilazane was slowly pumped into the autoclave. Thereafter, the autoclave was heated to 80° C. and maintained for 3 hours. The other operations were the same as that in Example 1. Finally, 36.8 g of hydrogen bis(fluorosulfonyl)imide was obtained, with a yield of 87%.

### Example 3

100 mL of anhydrous ethyl acetate and 76.5 g of sulfonyl fluoride were added to a 500-mL autoclave. At room temperature, 40.35 g of hexamethyl disilazane was slowly pumped into the autoclave. Thereafter, the autoclave was heated to 100° C. and maintained for 3 hours. The other operations were the same as that in Example 1. Finally, 42.01 g of hydrogen bis(fluorosulfonyl)imide was obtained, with a yield of 95%.

### Example 4

125 mL of anhydrous dimethyl carbonate and 6 g of lithium fluoride were added to a 200-mL three-necked flask. The flask was cooled to 0° C. 36.4 g of HFSI obtained in Example 1 was dropwise added to the flask at the temperature less than 5° C. Thereafter, the mixture in the flask was maintained at 0° C. for 3 hours. The mixture was filtered. Unreacted lithium fluoride was removed, and the supernatant was concentrated to 58 g. 125 g of dichloroethane was mixed with the supernatant, and a white solid was precipitated. The white solid was filtered and dried, thereby yield LiFSI.

### Example 5

130 mL of anhydrous ether and 6 g of lithium hydroxide were added to a 200-mL three-necked flask. The flask was cooled to 0° C. 36.8 g of HFSI obtained in Example 2 was dropwise added to the flask at the temperature less than 5° C. Thereafter, the mixture in the flask was maintained at 0° C. for 2 hours. The mixture was filtered. Unreacted lithium hydroxide was removed, and the supernatant was concentrated to 60 g. 130 g of dichloromethane was mixed with the supernatant, and a white solid was precipitated. The white solid was filtered and dried, thereby yield LiFSI.

### Example 6

125 mL of anhydrous ethyl acetate and 9.96 g of lithium carbonate were added to a 300-mL three-necked flask. The flask was cooled to 0° C. 42.01 g of HFSI obtained in Example 3 was dropwise added to the flask at the temperature less than 5° C. Thereafter, the mixture in the flask was maintained at 0° C. for 3 hours. The mixture was filtered. Unreacted lithium carbonate was removed, and the supernatant was concentrated to 68 g. 140 g of n-hexane was mixed with the supernatant, and a white solid was precipitated. The white solid was filtered and dried, thereby yield LiFSI.

The preparation method for LiFSI comprises contacting sulfonyl fluoride with hexamethyl disilazane to yield HFSI; the HFSI reacts with lithium to yield LiFSI with a high purity. The byproduct trimethylfluorosilane can react with ammonia gas to yield hexamethyl disilazane for recycling. The method is easy to operate and is cost-effective.

It will be obvious to those skilled in the art that changes and modifications may be made, and therefore, the aim in the appended claims is to cover all such changes and modifications.

What is claimed is:

1. A method, comprising contacting sulfonyl fluoride with hexamethyl disilazane in an organic solvent, a reaction process being as follows:

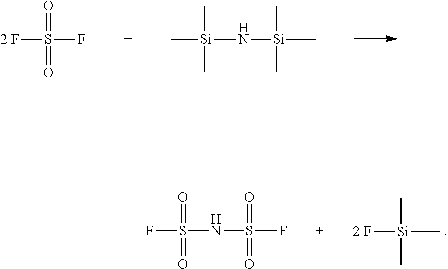

2. The method of claim 1, wherein the organic solvent is an ester, an amide, or a nitrile; the ester comprises ethyl acetate and butyl acetate; the amide comprises N, N-dimethylformamide, N, N-Dimethylacetamide, and N-methylpyrrolidone; and the nitrile comprises acetonitrile and propiononitrile.

3. The method of claim 1, wherein the method comprises dissolving the sulfonyl fluoride in the organic solvent, and adding the hexamethyl disilazane to a mixture of the sulfonyl fluoride and the organic solvent; and an addition amount of the organic solvent is at least 0.1 L per mole of hexamethyl disilazane.

4. The method of claim 2, wherein the method comprises dissolving the sulfonyl fluoride in the organic solvent, and adding the hexamethyl disilazane to a mixture of the sulfonyl fluoride and the organic solvent; and an addition amount of the organic solvent is at least 0.1 L per mole of hexamethyl disilazane.

5. The method of claim 1, wherein contacting the sulfonyl fluoride with the hexamethyl disilazane is carried out at a temperature of between 30 and 110° C. for 2-10 hours.

6. The method of claim 2, wherein contacting the sulfonyl fluoride with the hexamethyl disilazane is carried out at a temperature of between 30 and 110° C. for 2-10 hours.

7. The method of claim 1, wherein a molar ratio of the sulfonyl fluoride to the hexamethyl disilazane is between 2:1 and 5:1.

8. The method of claim 2, wherein a molar ratio of the sulfonyl fluoride to the hexamethyl disilazane is between 2:1 and 5:1.

9. The method of claim 1, wherein a byproduct of trimethylfluorosilane is produced, and the method further comprises contacting the trimethylfluorosilane with ammonia gas to yield the hexamethyl disilazane, a reaction process being as follows:

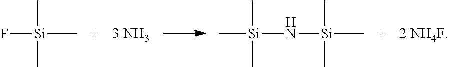

10. A method, comprising:
contacting sulfonyl fluoride with hexamethyl disilazane in an organic solvent, thereby yielding hydrogen bis(fluorosulfonyl)imide (HFSI); and
contacting hydrogen bis(fluorosulfonyl)imide with a lithium compound, thereby yielding lithium bis(fluorosulfonyl)imide (LiFSI).

11. The method of claim 10, wherein the lithium compound is selected from the group consisting of Li, LiH, LiNH₂, LiF, LiOH, LiHCO₃, Li₂CO₃, or a mixture thereof.

12. The method of claim 10, wherein the organic solvent is a polar solvent selected from the group consisting of dimethyl carbonate, diethyl carbonate, methylethyl carbonate, propylene carbonate, vinyl carbonate, methyl acetate, propyl acetate, isopropyl acetate, ethyl acetate, butyl acetate, isobutyl acetate, ether, propyl ether, isopropyl ether, butyl ether, isobutyl ether, tetrahydrofuran, methyltetrahydrofuran, dioxane, ethylene glycol dimethyl ether, ethylene glycol diethyl ether, acetone, butanone Methyl isobutyl ketone, cyclopentanone, cyclobutanone, N, N-dimethylformamide, N, N-Dimethylacetamide, N-methylpyrrolidone, dimethyl sulfoxide, acetonitrile, propiononitrile, or a mixture thereof; and an addition amount of the organic solvent is at least 0.1 L per mole of hexamethyl disilazane.

13. The method of claim 11, wherein the organic solvent is a polar solvent selected from the group consisting of dimethyl carbonate, diethyl carbonate, methylethyl carbonate, propylene carbonate, vinyl carbonate, methyl acetate, propyl acetate, isopropyl acetate, ethyl acetate, butyl acetate, isobutyl acetate, ether, propyl ether, isopropyl ether, butyl ether, isobutyl ether, tetrahydrofuran, methyltetrahydrofuran, dioxane, ethylene glycol dimethyl ether, ethylene glycol diethyl ether, acetone, butanone Methyl isobutyl ketone, cyclopentanone, cyclobutanone, N, N-dimethylformamide, N, N-Dimethylacetamide, N-methylpyrrolidone, dimethyl sulfoxide, acetonitrile, propiononitrile, or a mixture thereof; and an addition amount of the organic solvent is at least 0.1 L per mole of hexamethyl disilazane.

14. The method of claim 10, wherein a molar ratio of the sulfonyl fluoride to lithium of the lithium compound is between 1:1 and 1:2.

15. The method of claim 11, wherein a molar ratio of the sulfonyl fluoride to lithium of the lithium compound is between 1:1 and 1:2.

16. The method of claim 10, wherein contacting hydrogen bis(fluorosulfonyl)imide with a lithium compound is carried out at a temperature of between 0 and 20° C. for 1-10 hours.

17. The method of claim 11, wherein contacting hydrogen bis(fluorosulfonyl)imide with a lithium compound is carried out at a temperature of between 0 and 20° C. for 1-10 hours.

18. The method of claim 10, comprising mixing the lithium compound and the organic solvent, cooling a mixture of the lithium compound and the organic solvent to between 0 and 2° C., dropwise adding hydrogen bis(fluorosulfonyl)imide to the mixture, resting the mixture and the hydrogen bis(fluorosulfonyl)imide at a temperature of between 0 and 2° C. for 1-5 hours, filtering and collecting a supernatant, concentrating the supernatant, adding a polar or nonpolar solvent to a resulting concentrated supernatant thereby yielding a solid lithium bis(fluorosulfonyl)imide, filtering and drying the solid lithium bis(fluorosulfonyl)imide.

19. The method of claim 11, comprising mixing the lithium compound and the organic solvent, cooling a mixture of the lithium compound and the organic solvent to between 0 and 2° C., dropwise adding hydrogen bis(fluorosulfonyl)imide to the mixture, resting the mixture and the hydrogen bis(fluorosulfonyl)imide at a temperature of between 0 and 2° C. for 1-5 hours, filtering and collecting a supernatant, concentrating the supernatant, adding a polar or nonpolar solvent to a resulting concentrated supernatant thereby yielding a solid lithium bis(fluorosulfonyl)imide, filtering and drying the solid lithium bis(fluorosulfonyl)imide.

20. The method of claim 18, wherein the polar or nonpolar solvent is a halogenated hydrocarbon solvent, alkane solvent, halogenated aromatic hydrocarbon solvent; the halogenated hydrocarbon solvent comprises dichloromethane and dichloroethane; the alkane solvent comprises n-hexane, cyclohexane and n-heptane, and the halogenated aromatic hydrocarbon solvent comprises toluene, ethylbenzene and chlorobenzene; and an addition amount of the polar or nonpolar solvent is 1-5 times that of the solid lithium bis(fluorosulfonyl)imide by weight.
